(19) United States
(12) Patent Application Publication
(10) Pub. No.: US 2015/0375228 A1
(43) Pub. Date: Dec. 31, 2015

(54) DEVICES FOR SORTING CELLS IN A SAMPLE AND METHODS FOR USE THEREOF

(71) Applicant: THE REGENTS OF THE UNIVERSITY OF CALIFORNIA, Oakland, CA (US)

(72) Inventors: Lydia L. SOHN, Oakland, CA (US); Matthew CHAMBERS, Berkeley, CA (US)

(21) Appl. No.: 14/765,103

(22) PCT Filed: Mar. 12, 2014
(86) PCT No.: PCT/US14/24574

§ 371 Date: Jul. 31, 2015

Related U.S. Application Data
(60) Provisional application No. 61/790,762, filed on Mar. 15, 2013.

(51) Int. Cl. B01L 3/00; C12Q 1/24

(52) U.S. Cl. CPC B01L 3/502761; C12Q 1/24; B01L 2200/0652

(57) ABSTRACT

Devices for sorting cells in a fluid sample are provided. The device includes a microfluidic conduit configured to carry a flow of a fluid sample. The conduit includes a longitudinal divider extending from an interior surface of the conduit towards an opposing interior surface of the conduit, where the longitudinal divider defines a first longitudinal fluid flow path in fluid communication with a second longitudinal fluid flow path in the conduit. Also provided are methods of using the devices as well as systems and kits that include the devices. The devices, systems and methods find use in a variety of different applications, including diagnostic assays.

### CROSS-REFERENCE TO RELATED APPLICATION

Pursuant to 35 U.S.C. §119(e), this application claims priority to the filing date of U.S. Provisional Application Ser. No. 61/790,762 filed on Mar. 15, 2013, the disclosure of which is incorporated herein by reference.

### INTRODUCTION

Cell characterization is used in cell biology for disease diagnosis and monitoring, and drug discovery. Although current methods for cell analysis, such as flow cytometry and magnetic-bead column selection have been used in both research laboratories and clinical settings, improved devices and methods may be desirable. For example, traditional approaches often require advanced preparation, including exogenous labeling of cells. Such labeling leads to added incubation time, additional costs, loss of sample, and the possibility of modifying cell physiology and function. In addition, traditional approaches do not lend themselves to portability, which can be desirable in certain clinical situations.

Circulating tumor cells (CTCs) are cells that have been shed from primary solid tumors and have entered into the blood stream. They are believed to play a key role in the metastatic progression of breast cancer, and as such, their very presence and, in particular, their number in a given volume of patient blood could provide a means for determining patient prognosis and for monitoring the progression of disease. Clinical studies have shown that breast-cancer patients with >5 CTCs in 7.5 mL of whole blood prior to the start of therapy had a shorter progression-free survival and poor overall survival than those with fewer to no CTCs in the same volume of blood. While the clinical and prognostic significance of CTCs continue to be established for metastatic breast cancer, very little is known about CTCs, themselves. Their isolation and classification are extremely difficult because they are extremely rare: 1-10 cells/7.5 mL of peripheral blood.

### SUMMARY

Devices for sorting cells in a fluid sample are provided. The device includes a microfluidic conduit configured to carry a flow of a fluid sample. The conduit includes a longitudinal divider extending from an interior surface of the conduit towards an opposing interior surface of the conduit, where the longitudinal divider defines a first longitudinal fluid flow path in fluid communication with a second longitudinal fluid flow path in the conduit. Also provided are methods of using the devices as well as systems and kits that include the devices. The devices, systems and methods find use in a variety of different applications, including diagnostic assays.

Aspects of the present disclosure include a device for sorting cells in a fluid sample. The device includes a microfluidic conduit configured to carry a flow of a fluid sample. The microfluidic conduit includes a longitudinal divider extending from an interior surface of the conduit towards an opposing interior surface of the conduit, where the longitudinal divider defines a first longitudinal fluid flow path in fluid communication with a second longitudinal fluid flow path in the conduit.

In some embodiments, the conduit includes a gap between the longitudinal divider and the opposing interior surface of the conduit.

In some embodiments, the gap has a height ranging from 1 μm to 50 μm.

In some embodiments, the first fluid flow path has a width that decreases along the length of the conduit.

In some embodiments, the second fluid flow path has a width that increases along the length of the conduit.

In some embodiments, the conduit includes an inlet in fluid communication with an upstream end of the first fluid flow path.

In some embodiments, the conduit includes a first outlet in fluid communication with a downstream end of the first fluid flow path.

In some embodiments, the conduit includes a second outlet in fluid communication with a downstream end of the second fluid flow path.

In some embodiments, the interior surface of the conduit includes one or more protrusions extending into the first fluid flow path.

Aspects of the present disclosure include a multiplex device for sorting cells in a fluid sample. The device includes a microfluidic conduit configured to carry a flow of a fluid sample. The microfluidic conduit includes two or more longitudinal dividers each extending from an interior surface of the conduit towards an opposing interior surface of the conduit, where the longitudinal dividers define three or more longitudinal fluid flow paths in fluid communication with each other in the conduit.

In some embodiments, the conduit includes a first gap between a first longitudinal divider and the opposing interior surface of the conduit, and a second gap between a second longitudinal divider and the opposing interior surface of the conduit.

In some embodiments, the first gap has a height greater than the second gap.

In some embodiments, the conduit includes an inlet in fluid communication with an upstream end of a first fluid flow path.

In some embodiments, the conduit includes one or more separate fluid outlets each in fluid communication with a downstream end of a different fluid flow path.

Aspects of the present disclosure include a method of sorting cells in a fluid sample. The method includes passing a fluid sample that includes a plurality of cells through a microfluidic conduit having a longitudinal divider extending from an interior surface of the conduit towards an opposing interior surface of the conduit, where the longitudinal divider defines a first longitudinal fluid flow path in fluid communication with a second longitudinal fluid flow path in the conduit.

In some embodiments, the method includes collecting a first population of cells from a first outlet in fluid communication with a downstream end of the first fluid flow path.

In some embodiments, the method includes collecting a second population of cells from a second outlet in fluid communication with a downstream end of the second fluid flow path.

In some embodiments, the method includes quantifying the cells that pass through the conduit.

In some embodiments, the method includes characterizing the cells that pass through the conduit.

Aspects of the present disclosure include a kit that includes a device that includes a microfluidic conduit configured to carry a flow of a fluid sample. The microfluidic conduit includes a longitudinal divider extending from an interior surface of the conduit towards an opposing interior surface of the conduit, where the longitudinal divider defines a first longitudinal fluid flow path in fluid communication with a second longitudinal fluid flow path in the conduit. The kit further includes a buffer.

### BRIEF DESCRIPTION OF THE FIGURES

FIG. 1(a) shows a weir device schematic (not to scale), according to embodiments of the present disclosure. FIG. 1(a) (left) shows a cross-sectional view of the weir, gap height, and basic operation. While white blood cells (WBCs) can deform and pass under the weir, MCF-7 cells cannot. The flow direction is into the page (circle/cross symbol). FIG. 1(a) (right) shows a top view of the device. Arrows indicate flow direction. FIG. 1(b) shows images of a weir device (33 μm×300 μm×3000 μm, H×W×L; gap height=9.0 μm, flow rate 1 μL/min) in operation. Images were taken at 1 sec. intervals. Periodic restrictions in the channel direct the cells toward the weir. However, while an MCF-7 cell only flows along the weir, a WBC passes underneath the weir to the other side of the channel.

FIG. 2 shows a cross-sectional schematic of a device according to embodiments of the present disclosure.

FIG. 3 shows a schematic view of a device that includes two weir divisions and three microchannels, according to embodiments of the present disclosure.

FIG. 4(a)-4(e) show: FIG. 4(a), a straight channel with a single weir; FIG. 4(b) a device with a tapered channel; FIG. 4(c) a device with alternating sections of pinched flow and un-pinched flow; FIG. 4(d) shows a device with two weirs; and FIG. 4(e) shows a device with two weirs with wedges in the circulating tumor cell (CTC) channel to direct blood cells toward the filter and to disturb stream lines, according to embodiments of the present disclosure.

FIG. 5(a) shows a device configured with an elongated channel to increase population purity in respective microchannels, according to embodiments of the present disclosure. FIG. 5(b) shows an embodiment of an elongated device with a serpentine conduit, according to embodiments of the present disclosure.

FIG. 6(a) shows an embodiment of an elongated device with a serpentine conduit, and FIG. 6(b) shows an embodiments of an elongated device with a serpentine conduit with a first flow path that tapers along its length, according to embodiments of the present disclosure.

FIG. 7 shows an image of a polydimethylsiloxane (PDMS) based device according to embodiments of the present disclosure.

FIG. 8 shows images taken over time of a device in operation, according to embodiments of the present disclosure.

FIG. 9 shows examples of process flow diagrams for the fabrication of devices, according to embodiments of the present disclosure.

FIG. 10(a) shows an image of a device composed of thermoset polyester (TPE), and FIG. 10(b) shows an image of a device composed of Norland optical adhesive (NOA), according to embodiments of the present disclosure.

FIG. 11 (top) shows a schematic drawing of a device that includes two weirs arranged in series, according to embodiments of the present disclosure. FIG. 11 (bottom) is an enlargement of the device.

### DETAILED DESCRIPTION

Devices for sorting cells in a fluid sample are provided. The device includes a microfluidic conduit configured to carry a flow of a fluid sample. The conduit includes a longitudinal divider extending from an interior surface of the conduit towards an opposing interior surface of the conduit, where the longitudinal divider defines a first longitudinal fluid flow path in fluid communication with a second longitudinal fluid flow path in the conduit. Also provided are methods of using the devices as well as systems and kits that include the devices. The devices, systems and methods find use in a variety of different applications, including diagnostic assays.

Before the present invention is described in greater detail, it is to be understood that aspects of the present disclosure are not limited to the particular embodiments described, and as such may, of course, vary. It is also to be understood that the terminology used herein is for the purpose of describing particular embodiments only, and is not intended to be limiting, since the scope of embodiments of the present disclosure will be defined only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within embodiments of the present disclosure. The upper and lower limits of these smaller ranges may independently be included in the smaller ranges and are also encompassed within embodiments of the present disclosure, subject to any specifically excluded limit in the stated range. Where the stated range includes one or both of the limits, ranges excluding either or both of those included limits are also included in embodiments of the present disclosure.

Unless defined otherwise, all technical and scientific terms used herein have the same meaning as commonly understood by one of ordinary skill in the art to which this invention belongs. Although any methods and materials similar or equivalent to those described herein can also be used in the practice or testing of embodiments of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that embodiments of the present disclosure are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of embodiments of the present disclosure, embodiments of the devices for detecting a particle in a fluid sample are described first in greater detail. Following this description, methods of detecting a particle in a fluid sample, followed by a description of embodiments of systems that include the devices are provided. Finally, a review of the various applications in which the devices, methods, and systems may find use is provided.

### Devices

### Conduit

In certain embodiments, devices of the present disclosure include a conduit configured to carry a flow of a fluid sample. Embodiments of the conduit, as well as other aspects of the devices are described in more detail below. In certain embodiments, the conduit is configured to carry a flow of a fluid sample. In certain embodiments, the conduit includes a longitudinal divider extending from an interior surface of the conduit towards an opposing interior surface of the conduit, where the longitudinal divider defines a first longitudinal fluid flow path in fluid communication with a second longitudinal fluid flow path in the conduit.

In certain embodiments, the conduit has a diameter of 0.1 μm to 1000 μm, such as 1 μm to 750 μm, including 10 μm to 500 μm. For example, the conduit may have a diameter of 500 μm. Although described in terms of diameter for circular cross sections, the dimensions described herein also apply to square or rectangular cross sectional conduits, such as the width and/or height of a square or rectangular cross section. In some cases, the conduit has a cross sectional area of 1 mm² or less, or 0.75 mm² or less, or 0.5 mm² or less, or 0.25 mm² or less, or 0.1 mm² or less. For instance, the conduit may have a cross sectional area of 0.25 mm² or less. The length of the conduit will generally be greater than its diameter (e.g., the conduit is an elongated conduit). In some instances, the length of the conduit is 1 to 1000 mm, or 1 to 750 mm, or 1 to 500 mm, or 1 to 250 mm, 1 to 100 mm, 1 to 50 mm, such as from 1 to 25 mm, or from 1 to 10 mm, or from 1 to 5 mm. In certain instances, the ratio of the length of the conduit to the diameter is 1000:1, such as 750:1, including 500:1, or 250:1, or 100:1, or 75:1, or 50:1, or 25:1 or 10:1.

Although the dimensions of the conduit have been described above, the dimensions of conduit may vary as desired with the size and shape of the cells to be sorted. The cross sectional profile of the conduit may be circular, square or rectangular. In certain embodiments, the cross sectional profile of the conduit is rectangular. However, for some applications, it may be desired to use other conduit shapes.

In certain embodiments, the conduit includes one or more longitudinal dividers in the conduit. By “divider” or “weir” is meant a wall that extends from a first interior surface of the conduit towards a second interior surface of the conduit. In some instances, the first interior surface of the conduit is opposite the second interior surface of the conduit. For example, the first and second interior surfaces may be opposing interior surfaces of the conduit. In certain cases, the first and second interior surfaces of the conduit are planar. In these embodiments, the first and second interior surfaces of the conduit may be substantially parallel to each other.

The divider may extend partway from the first interior surface to the second interior surface. For instance, the divider may extend from the first interior surface but not extend completely across the interior of the conduit to contact the second interior surface of the conduit. As such, the conduit may include a gap between the divider and the second interior surface of the conduit. For example, the conduit may include a gap between a top surface of the divider and the opposing second interior surface of the conduit. By “gap” is meant a void through which fluid and/or cells may flow. In some instances, the gap between the divider and the second interior surface of the conduit runs along the entire length of the divider, such that the divider does not contact the second interior surface of the conduit along its length.

In certain embodiments, the gap has a height ranging from 1 μm to 50 μm, such as from 1 μm to 40 μm, including from 1 μm to 30 μm, or from 1 μm to 25 μm, or from 1 μm to 20 μm, or from 1 μm to 15 μm, or from 1 μm to 10 μm, or from 1 μm to 5 μm. In some instances, the gap has a height of 10 μm. In some instances, the gap has a height of 10 μm or less, such as 9 μm, or 8 μm, or 7 μm, or 6 μm, or 5 μm, or 4 μm, or 3 μm, or 2 μm or less. In certain instances, the gap has a height of 9 μm. In certain instances, the gap has a height of 3.75 μm.

In certain embodiments, the longitudinal divider is positioned at any position(s) along the conduit, such that the longitudinal divider extends along substantially the entire length of the conduit (e.g., substantially parallel to the fluid flow path through the conduit). In certain embodiments, the divider defines two or more fluid flow paths within the conduit. For example, an embodiment of the conduit may include one longitudinal divider, and as such the longitudinal divider may define a first fluid flow path and a second fluid flow path within the conduit. The first and second fluid flow paths may be in fluid communication with each other via the gap between the divider and the interior surface of the conduit described above. As such, the divider may be positioned between adjacent fluid flow paths of the conduit. For example, the divider may be flanked on each side by a fluid flow path of the conduit.

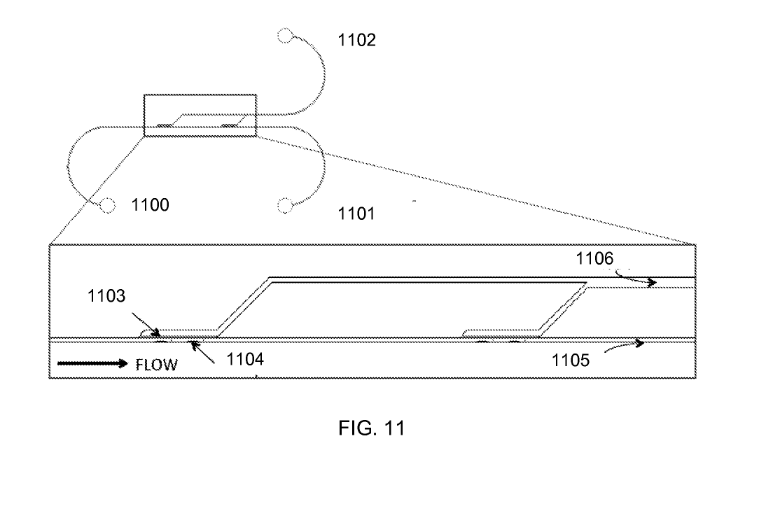

In certain embodiments, the device includes two or more dividers in the conduit. For example, each divider may have a length shorter than the length of the conduit and the dividers may be arranged in the conduit in series, such that fluid flowing through the conduit contacts the dividers sequentially. In some embodiments, the inter-divider portions of the fluid flow path may be substantially parallel to the divider. In some embodiments, the inter-divider portions of the fluid flow path may be separated from the initial fluid flow path, e.g., a portion of the inter-divider portions of the second fluid flow path may have a longitudinal axis at an angle (such as an angle from 0.5 to 90 degrees) to the longitudinal axis of the first fluid flow path. See, e.g., FIG. 11. In some embodiments, each divider may have a length substantially the same as the length of the conduit and the dividers may be arranged in the conduit in parallel. In some embodiments, the conduit may include a plurality of dividers in the conduit where the dividers are arranged in a series of parallel dividers. In some instances, the conduit includes 2 or more dividers, such as 3 or more dividers, or 4 or more, or 5 or more, or 6 or more, or 7 or more, or 8 or more, or 9 or more, or 10 or more dividers. The dividers in the conduit may be the same (e.g., the dividers may have the same gap height), or the dividers may be different (e.g., the dividers may have different gap heights). Embodiments where the dividers have different gap heights may facilitate the separation of different populations of cells from a sample flowing through the device. For example, the conduit may include a series of dividers, with the dividers having progressively larger gap heights along the fluid flow path, e.g., the first divider has a gap height less than the gap height of the second divider, which has a gap height less than the gap height of the third divider (if present), which has a gap height less than the gap height of the fourth divider (if present), etc.

An embodiment of a device that includes a series of dividers is shown in FIG. 11. FIG. 11 (top) shows a schematic drawing of a device that includes two dividers arranged in series. The device includes a fluid inlet (1100) in fluid communication with an upstream end of a first fluid flow path, a first fluid outlet (1101) in fluid communication with a downstream end of the first fluid flow path, and a second fluid outlet (1102) in fluid communication with a downstream end of a second fluid flow path. FIG. 11 (bottom) shows an enlarged view of the device. The device includes a first divider (1103) between the first fluid flow path and the second fluid flow path, and protrusions (1104) in the first fluid flow path that direct cells in the fluid flow towards the divider. A first cell population can be collected from an output of the first fluid flow path (1105), and a second population of cells can be collected from an output of the second fluid flow path (1106).

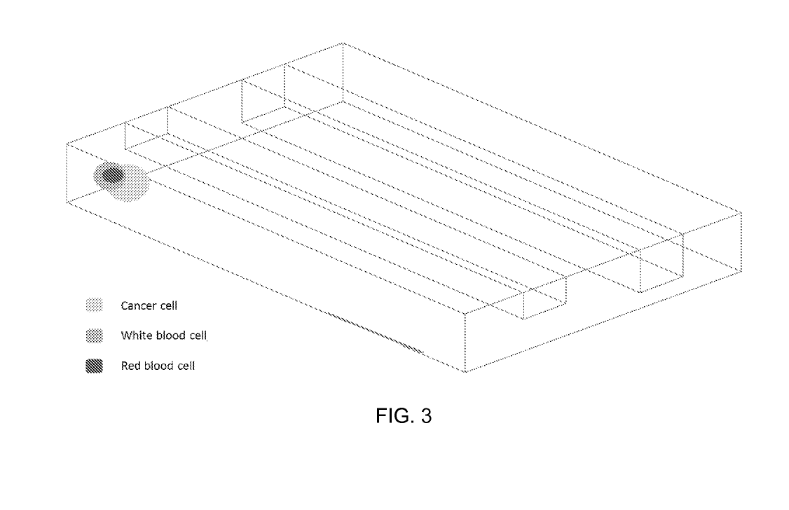

For example, in some instances, the conduit includes two dividers arranged in parallel, which divide the conduit into three fluid flow paths. The gap for each divider may be different from each other, such that different populations of cells are retained in each of the three fluid flow paths. For example, for circulating tumor cell (CTC) isolation, individual fluid flow paths may preferentially retain leukocytes, erythrocytes and CTCs, respectively. FIG. 3 illustrates an embodiment of a device that includes a conduit with two different dividers, e.g., the conduit includes a first divider and a second divider, where the gap height of the first divider is greater than the gap height of the second divider.

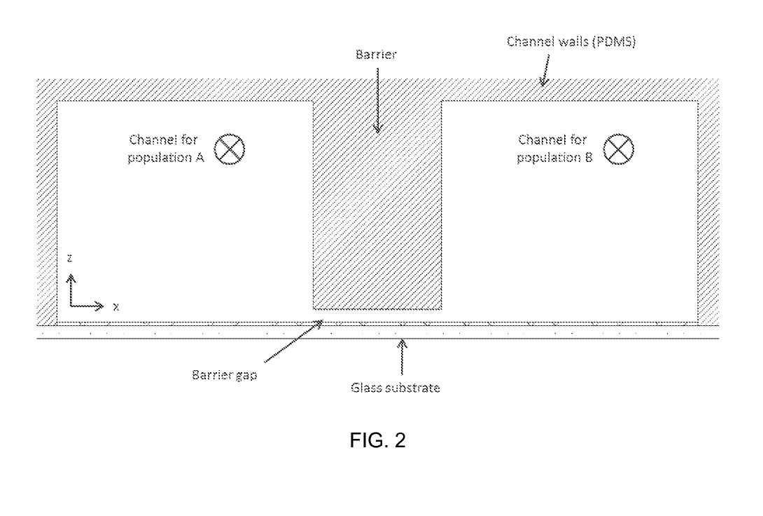

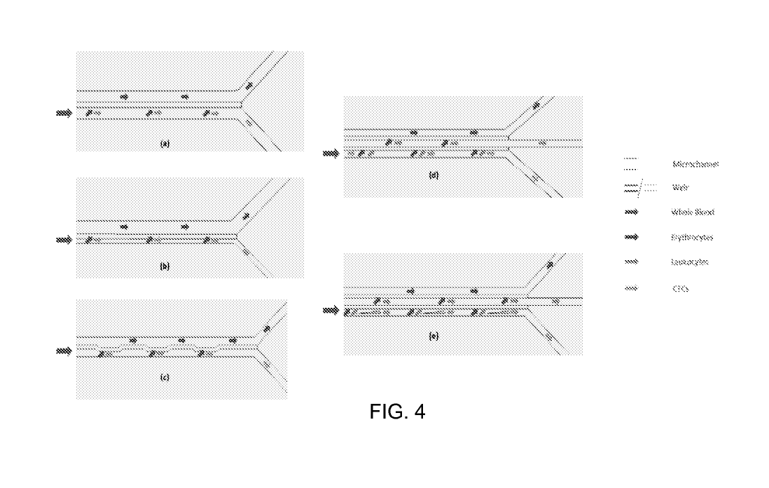

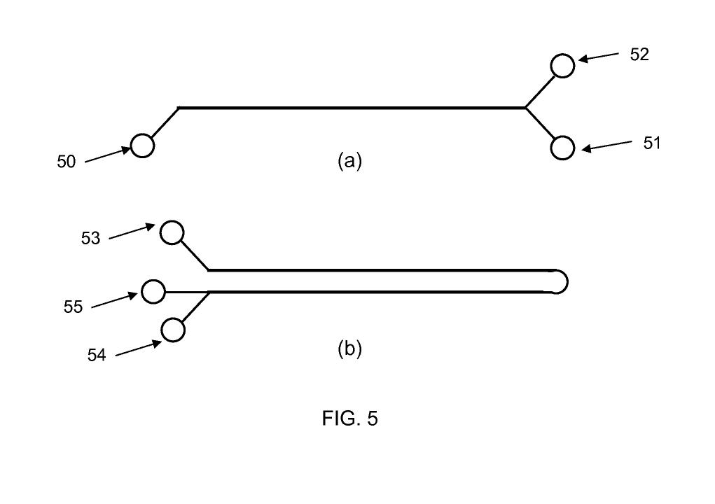

FIG. 4(a) shows a schematic drawing of a conduit with a single divider. Embodiments such as those illustrated in FIG. 4(a) can be used to separate two cell populations, for example, CTCs can be isolated from a sample of CTCs and erythrocytes. FIG. 2 shows a cross-sectional schematic of a device that includes a conduit with a single divider. FIGS. 4(b) and 4(c) show devices designed to remove a first population of cells (e.g., erythrocytes) by constricting the input channel and forcing cells to interact with the divider. In FIG. 4(b) the conduit is tapered; e.g., 100 microns at the upstream end of the conduit and 50 microns at the downstream end. In FIG. 4(c) the conduit includes alternating sections having a smaller dimension (e.g., conduit width). For example, the conduit may include a series of protrusions that direct cells flowing through the conduit towards the divider. FIGS. 4(d) and 4(e) show devices with two dividers that illustrate the separation of CTCs, leukocytes, and erythrocytes. The dividers shown in FIGS. 4(d) and 4(e) have different gap heights. FIG. 4(e) includes protrusions in the initial fluid flow path of the conduit to direct cells toward the divider. The devices shown in FIG. 4 have arrows to represent the directions in which cell populations move within the device. Any number of dividers can be included in the conduit to sort different cell populations. In addition, the device length can be increased as shown in FIGS. 5(a)-5(b) to any desired length. FIG. 5(a) shows a device configured with an elongated linear conduit. The device shown in FIG. 5(a) includes a fluid inlet (50) in fluid communication with an upstream end of a first fluid flow path, a first fluid outlet (51) in fluid communication with a downstream end of the first fluid flow path, and a second fluid outlet (52) in fluid communication with a downstream end of a second fluid flow path. In some instances, a longer conduit may facilitate an increase in cell population purity in the respective fluid flow paths. FIG. 5(b) shows an embodiment of an elongated device with a serpentine conduit. The device shown in FIG. 5(b) includes a fluid inlet (53) in fluid communication with an upstream end of a first fluid flow path, a first fluid outlet (54) in fluid communication with a downstream end of the first fluid flow path, and a second fluid outlet (55) in fluid communication with a downstream end of a second fluid flow path.

During use, a fluid sample may be passed through the conduit (e.g., flow through the conduit). In some instances, the fluid sample includes one or more populations of cells. For example, the fluid sample may include a first population of cells and a second population of cells. The first and second population of cells may be different from each other based on one or more physical characteristics of the cells. For instance, the cell populations may differ based on average cell size (e.g., diameter). By “average” is meant the arithmetic mean. In some instances, the cell populations may differ based on the deformability of the cells. As the fluid sample flows through the conduit, cells in the first population of cells may be retained in the first fluid flow path while cells in the second population of cells may traverse the divider between the first fluid flow path and the second fluid flow path, such that cells of the second population enter and are retained within the second fluid flow path. For example, cells in the first population may have an average diameter that is larger than the gap height of the divider, or cells in the second population may have an average diameter less than the gap height, or cells in the first population may not be deformable to an extent necessary to pass through the gap between the first and second flow paths, or the cells in the second population may be deformable to an extent sufficient for the cells to pass through the gap between the first and second flow paths, or a combination of one or more of the above.

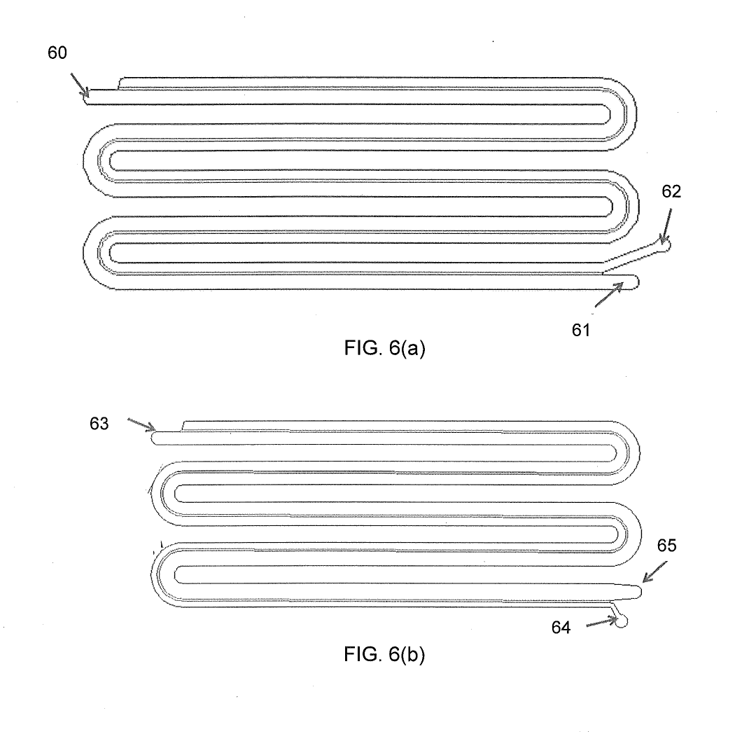

In certain embodiments, the conduit includes a first fluid flow path where the first fluid flow path has a width that decreases along the length of the conduit. In some instances, the conduit includes a second flow path where the second fluid flow path has a width that increases along the length of the conduit. As such, in some cases, the overall diameter (or dimensions) of the conduit may be substantially the same along the length of the conduit. FIG. 6(a) shows an embodiment of an elongated device with a serpentine conduit. The device shown in FIG. 6(a) includes a fluid inlet (60) in fluid communication with an upstream end of a first fluid flow path, a first fluid outlet (61) in fluid communication with a downstream end of the first fluid flow path, and a second fluid outlet (62) in fluid communication with a downstream end of a second fluid flow path. FIG. 6(b) shows an embodiment of an elongated device with a serpentine conduit, where the first fluid flow path has a width that decreases along its length and the second fluid flow path has a width that increases along it length. The device shown in FIG. 6(b) includes a fluid inlet (63) in fluid communication with an upstream end of a first fluid flow path, a first fluid outlet (64) in fluid communication with a downstream end of the first fluid flow path, and a second fluid outlet (65) in fluid communication with a downstream end of a second fluid flow path. For instance, the width of the second fluid flow path may increase by about 200 μm for each linear section of the conduit. Similarly, the width of the first fluid flow path may decrease by about 200 μm for each linear section of the conduit.

In certain embodiments, the conduit includes an inlet at the upstream end of the conduit. In certain embodiments, the conduit includes a single inlet at the upstream end of the conduit. By “upstream” is meant at a position nearer to the source of the fluid flow. By “downstream” is meant at a position further away from the source of the fluid flow. In some instances, the conduit includes an inlet in fluid communication with an upstream end of the first fluid flow path. The fluid sample may be introduced into the conduit through the inlet, and as the sample flows through the conduit, the cells may be sorted based on one or more physical characteristics of the cells as described above.

In certain embodiments, the conduit includes at least one, and in some instances a plurality, of fluid outlets at a downstream end of the conduit. In some cases, the conduit includes a first outlet in fluid communication with a downstream end of the first fluid flow path. In some instances, the conduit includes a second outlet in fluid communication with a downstream end of the second fluid flow path. As such, cells of a first population that are retained in the first fluid flow path may be collected at the first outlet, and cells of a second population that traverse the divider (e.g., cells that pass through the gap between the divider and the interior surface of the conduit) and are retained in the second fluid flow path may be collected at the second outlet separately from the first population of cells.

In certain embodiments, the conduit has an interior surface that has one or more protrusions extending into the first fluid flow path. The protrusions may extend from an interior surface of the first fluid flow path towards the longitudinal divider. In certain embodiments, the protrusions extend from a surface other than the first interior surface (e.g., the surface from which the divider extends) and the opposing second interior surface as described above. For example, the protrusions may extend from an interior surface of the conduit that faces a side surface of the divider. Stated another way, the conduit may include a third interior surface (e.g., different from the first and second interior surfaces described above), where the third interior surface joins the first and second interior surfaces and opposes a side surface of the divider. The protrusions may extend from the third interior surface towards the opposing side surface of the divider. In certain embodiments, the third interior surface (and the protrusions thereon) are part of the first fluid flow path. In some instances, the third interior surface is substantially perpendicular to the first and second interior surfaces (e.g., where the first and second interior surfaces are substantially parallel).

The protrusions may be configured to direct the flow of the fluid (e.g., and the cells therein) in the first fluid flow path towards the longitudinal divider. In some instances, directing the flow of the fluid towards the divider may facilitate sorting of the different populations of cells in the fluid sample. In certain embodiments, the protrusions do not extend all the way from the interior surface of the conduit to the longitudinal divider, such that the flow of cells through the conduit is not substantially impeded (e.g., the flow of cells retained within the first fluid flow path are not substantially impeded). For example, there may be a gap between the protrusions and the facing side of the divider, where the gap is of a sufficient distance to allow fluid and cells to flow through the first fluid flow path. The conduit may include 1 or more protrusions, such as 2 or more protrusions, or 3 or more protrusions, or 4 or more protrusions, such as 5 or more, including 10 or more, or 20 or more, 25 or more, or 30 or more, or 35 or more, or 40 or more, or 45 or more, or 50 or more, or 75 or more, or 100 or more protrusions. The protrusions may be positioned along the conduit at regular intervals such that the distance between each protrusion is substantially the same. In other cases, the protrusions may be positioned along the conduit such that the distance between adjacent protrusions is different.

In certain embodiments, a protrusion has a thickness (e.g., as measured from the interior surface of the conduit to the side of the protrusion that extends away from the interior surface and into the conduit) that varies along the length of the protrusion. In some instances, the protrusion has a thickness that increases along at least a portion of its length. For example, the protrusion may have a first end that is thinner than a second end of the protrusion, e.g., the protrusion, or a portion thereof, may have a wedge shape. In some instances, the protrusion, or a portion thereof, is wedge shaped and positioned in the conduit such that the thinner end of the wedge is the upstream end of the protrusion and the thick end of the wedge is the downstream end of the protrusion. In these embodiments, fluid and/or cells flowing through the conduit contact the thinner end of the protrusion first and may be directed towards the divider as the fluid and/or cells flow past the increasing thickness of the protrusion. In embodiments that include two or more protrusions, each protrusion may have the same shape and dimensions. In other embodiments, the protrusions may have different shapes and/or dimensions.

In certain embodiments, a protrusion has a length (e.g., the dimension of the protrusion along the length of the conduit) of 10 μm or more, such as 15 μm or more, or 20 μm or more, or 25 μm or more, or 30 μm or more, or 35 μm or more, or 40 μm or more, or 45 μm or more, or 50 μm or more, or 60 μm or more, or 70 μm or more, or 80 μm or more, or 90 μm or more, or 100 μm or more, or 125 μm or more, or 150 μm or more, or 175 μm or more, or 200 μm or more, or 250 μm or more, or 300 μm or more, or 350 μm or more, or 400 μm or more, or 450 μm or more, or 500 μm or more. In some instances, the protrusion has a length of 200 μm. In certain embodiments, the protrusion has a thickness (e.g., the dimension of the protrusion from the interior surface of the conduit from which the protrusion extends to the opposing side of the protrusion that extends into the interior of the conduit) ranging from 1 μm to 500 μm, such as 5 μm to 400 μm, or 10 μm to 300 μm, or 50 μm to 300 μm, or 50 μm to 200 μm, or 50 μm to 150 μm. In some instances, the protrusion has a thickness of 100 μm. In certain embodiments, the protrusion has a width (e.g., the dimension of the protrusion between the first and second interior surfaces of the conduit) equal to the distance between the first and second interior surfaces of the conduit.

In certain embodiments, the conduit is formed in a substrate. For instance, the conduit may be formed as a channel in the substrate. Suitable substrate materials are generally selected based upon their compatibility with the conditions present in the particular operation to be performed by the device. Such conditions can include various pH, temperature, ionic concentration, solvent tolerance and application of electric fields. In certain instances, the substrate material is inert to components of an analysis to be carried out by the device. For example, the substrate material may be selected such that the substrate material does not substantially react with the reagents, fluids and/or particles in the samples to be analyzed by the device. Suitable substrate materials include, but are not limited to, glass, quartz, ceramics, and silicon, semiconductor (InAs, GaAs, and the like), as well as polymeric substances, e.g., plastics, such as, but not limited to, polydimethylsiloxane (PDMS), thermoset polyester (TPE), Norland Optical Adhesive (NOA; e.g., NOA 81), and the like. NOA 81 is a mercapto-ester polymer. In some instances, NOA is polymerized by exposure to light, such as UV light (e.g., UV light with a wavelength from 320 nm to 380 nm, such as 365 nm).

In embodiments that include polymeric substrates, the substrate materials may be rigid, semi-rigid, or non-rigid, opaque, semi-opaque, or transparent, depending upon the use for which they are intended. For example, devices which include an optical or visual detection element may be used with substrates that include optically transparent materials to facilitate the optical or visual detection. Alternatively, optically transparent windows of glass or quartz, e.g., may be incorporated into the substrate for these types of detection. As used herein, “optically transparent” means that the material allows light of wavelengths ranging from 180 to 1500 nm, such as from 220 to 800 nm, including from 250 to 800 nm, to be transmitted through the material with low transmission losses. Such light transmissive polymeric materials may be characterized by low crystallinity and include, but are not limited to, polycarbonate, polyethylene terepthalate, polystyrene, polymethylpentene, fluorocarbon copolymers, polyacrylates (including polymethacrylates, such as polymethylmethacrylate (PMMA)), and the like. The polymeric materials may have linear or branched backbones, and may be crosslinked or non-crosslinked. Examples of polymeric materials include, e.g., polydimethylsiloxane (PDMS), polyurethane, polyvinylchloride (PVC), polystyrene, polysulfone, polycarbonate, polymethylmethacrylate (PMMA) and the like. In certain embodiments, the substrate includes polydimethylsiloxane (PDMS).

The substrate may additionally be provided with access ports and/or reservoirs for introducing the various fluid elements needed for a particular analysis, as well as outlets for eliminating the various fluids, as described in more detail below.

In certain embodiments, the conduit is formed in the substrate. For example, the substrate may be configured with a conduit passing through the substrate, or a portion of the substrate (e.g., through a central portion of the substrate). In some instances, the conduit may be formed by removing a portion of the substrate (e.g., by drilling, boring, punching, coring, etc. through the substrate). In other instances, the substrate may be formed using a mold that upon removal of the mold leaves a conduit formed through the substrate (or a portion of the substrate as described above). In certain instances, where the conduit is formed in the substrate itself, a cover (as described in more detail below) may not be needed.

### Cover

In certain embodiments, the device includes a cover that overlays the substrate to enclose and fluidically seal channels in the substrate to form the conduit and reservoirs. The cover also include access ports and/or reservoirs for introducing the various fluid elements needed for a particular analysis, as well as outlets for eliminating the various fluids.

The cover may be attached to the substrate by a variety of means, including, e.g., thermal bonding, adhesives, or in the case of certain substrates, e.g., quartz, glass, or polymeric substrates, a natural adhesion between the two components. In some instances, the cover includes an elastomeric material. For example, an elastomeric cover may form a reversible hermetic seal with a smooth planar substrate. Forming a seal in this manner between the substrate and the cover may facilitate removal of the cover from the substrate such that the substrate and the cover may be washed and re-used. Alternatively, the cover may be bonded to the substrate, forming a permanent bond. Forming a permanent bond between the substrate and the cover may facilitate sealing of the cover to the substrate when higher fluid pressures are used. Bonding methods may be used to secure the cover to the substrate, including activating the elastomer surface, for example by plasma exposure, so that the elastomeric cover will bond when placed in contact with the substrate. In certain cases, the cover and substrate are oxidized in a (DC- or AC-generated) oxygen plasma to increase the hydrophilicity of the conduit and to strengthen the seal to the substrate.

The cover may be made from an elastomer, such as, but not limited to, polyisoprene, polybutadiene, polychloroprene, polyisobutylene, poly(styrene-butadiene-styrene), polyurethane, and silicone. Polymers incorporating materials such as chlorosilanes or methyl-, ethyl-, and phenylsilanes, and polydimethylsiloxane (PDMS), or aliphatic urethane diacrylates may also be used. In some cases, the cover is made from materials, such as polyisoprene, polybutadiene, polychloroprene, polyisobutylene, poly(styrene-butadiene-styrene), the polyurethanes, and silicone polymers; or poly(bis(fluoroalkoxy)phosphazene) (PNF, Eypel-F), poly(carborane-siloxanes) (Dexsil), poly(acrylonitrile-butadiene) (nitrile rubber), poly(l-butene), poly(chlorotrifluoroethylene-vinylidene fluoride) copolymers (Kel-F), poly(ethyl vinyl ether), poly(vinylidene fluoride), poly(vinylidene fluoride-hexafluoropropylene) copolymer (Viton), and elastomeric compositions of polyvinylchloride (PVC), polysulfone, polycarbonate, polymethylmethacrylate(PMMA), and polytertrafluoroethylene (Teflon).

In certain embodiments, the thickness of the cover ranges from 0.1 μm to 10 cm, such as from 1 μm to 5 cm, including from 10 μm to 2 cm, or from 100 μm to 10 mm. In some cases, the cover has a thickness of 1 mm to 5 mm, such as 3 mm thick.

### Reservoirs

In certain embodiments, the device includes one or more reservoirs, such as one or more fluid reservoirs. The reservoirs may be configured to contain a fluid and/or direct the fluid to or from the conduit. For example, the device may include two reservoirs, such as a first reservoir and a second reservoir. The first reservoir may be in fluid communication with an end of the conduit, such as the upstream end of the conduit. The first reservoir may be configured to contain a fluid (e.g., a sample fluid), and direct the fluid to the upstream end of the conduit. The second reservoir may be in fluid communication with the other end of the conduit, such as the downstream end of the conduit. The second reservoir may be configured to contain the fluid exiting the downstream end of the conduit. Additional reservoirs may be provided, for instance in embodiments with two or more fluid outlets at the downstream end of the conduit, individual reservoirs may be provided at each of the separate fluid outlets.

In certain embodiments, the conduit is formed in the substrate as described above. For instance, the conduit may be formed as a channel in the substrate. Applying the cover to the substrate, as described above, may result in the formation of an enclosed conduit. Similarly, applying the cover to the substrate may result in two or more enclosed reservoirs in fluid communication with the conduit. Each of the reservoirs may be adapted to contain a fluid, such as a fluid sample that includes one or more cells. In certain cases, the reservoirs are configured to contain a fluid volume of 1 μL or more, such as 5 μL or more, including 10 μL or more, or 25 μL or more, or 50 μL or more, or 75 μL or more, or 100 μL or more, or 250 μL or more, or 500 μL or more, or 750 μL or more, or 1 mL or more.

In certain cases, the reservoir includes an inlet port. A fluid sample may be introduced into the reservoir through the inlet port. In certain instances, the reservoir includes an outlet port. A fluid can be transferred from the reservoir through the outlet port. A wide range of suitable sizes of inlets and outlets are possible, and those of skill in the art are capable of empirically determining the desired size ranges depending upon the nature of the fluid or the cells to be analyzed. In some cases, the reservoir includes a filter. For example, the reservoir positioned upstream from the conduit may include a filter configured to prevent large particles from clogging the conduit. In other cases, the device does not include a filter. For example, the size of the conduit may be large enough such that the risk of clogging is relatively low.

### Detector

In certain embodiments, the device includes a detector. The detector may be configured to detect cells that are passing through or have already passed through the conduit. For example, the detector may be configured to detect a first population of cells flowing through the first fluid flow path of the conduit. In some instances, the detector may be configured to detect a second population of cells flowing through the second flow path of the conduit. In certain embodiments, the detector is configured to quantify the cells. The detector may include a camera, complementary metal-oxide semiconductor (CMOS), charge-coupled device (CCD), intensified charge-coupled device (ICCD), fluorescence detector, combinations thereof, and the like.

### Methods

Aspects of the present disclosure include a method of sorting cells in a fluid sample. The method includes passing a fluid sample that includes a plurality of cells through a microfluidic conduit. As described above, the conduit includes a longitudinal divider extending from an interior surface of the conduit towards an opposing interior surface of the conduit, where the longitudinal divider defines a first longitudinal fluid flow path in fluid communication with a second longitudinal fluid flow path in the conduit.

Embodiments of the methods are directed to determining whether a population of cells is present in a sample, e.g., determining the presence or absence of one or more populations of cells in a sample. In certain embodiments of the methods, the presence of one or more populations of cells in the sample may be determined qualitatively or quantitatively. Qualitative determination includes determinations in which a simple yes/no result with respect to the presence of a population of cells in the sample is provided to a user. Quantitative determination includes both semi-quantitative determinations in which a rough scale result, e.g., low, medium, high, is provided to a user regarding the amount of cells in the sample, and fine scale results in which an exact measurement of the amount of the cells is provided to the user.

Samples that may be assayed with the subject microfluidic devices may vary, and include both simple and complex samples. Simple samples are samples that include the cells of interest, and may or may not include one or more molecular entities that are not of interest, where the number of these non-interest molecular entities may be low, e.g., 10 or less, 5 or less, etc. Simple samples may include biological or other samples that have been processed in some manner, e.g., to remove potentially interfering molecular entities from the sample. By “complex sample” is meant a sample that may or may not have the cells of interest, but also includes many different proteins, cells and/or other molecules that are not of interest. In some instances, the complex sample assayed in the subject methods is one that includes 10 or more, such as 20 or more, including 100 or more, e.g., 10³ or more, 10⁴ or more (such as 15,000; 20,000 or 25,000 or more) distinct (i.e., different) molecular entities, that differ from each other in terms of molecular structure or physical properties (e.g., size, deformability, shape, etc.).

In certain embodiments, the samples of interest are biological samples, such as, but not limited to, urine, blood, serum, plasma, saliva, semen, prostatic fluid, nipple aspirate fluid, lachrymal fluid, perspiration, feces, cheek swabs, cerebrospinal fluid, amniotic fluid, gastrointestinal fluid, biopsy tissue, cell lysate samples, and the like. The sample can be a biological sample or can be extracted from a biological sample derived from humans, animals, plants, fungi, yeast, bacteria, tissue cultures, viral cultures, or combinations thereof using conventional methods. In certain embodiments, the sample is a fluid sample, such as a solution of cells in a fluid. The fluid may be an aqueous fluid, such as, but not limited to water, saline, a buffer, and the like.

In certain instances, the sample is a whole blood sample. In some cases, red blood cells (RBCs) have a smaller average size than the cells of interest (e.g., metastatic cancer cells) and will pass through the gap and into the second fluid flow path. In some instances, different flow patterns may be used to remove RBCs and white blood cells (WBCs) from the fluid flow. In some cases, the RBCs may be lysed before or after isolation. In some cases, the device may be treated with bovine serum albumin (BSA) prior to flowing the sample through the device, which may facilitate a reduction in clogging of the device.

In certain instances, the method includes sorting the cells to determine the presence of the cells in the fluid sample. Because sorting of the cells of interest is based on physical characteristics of the cells, such as average size and/or deformability, (rather than, for example fluorescence-based detection techniques), in some embodiments, the cells are not labeled prior to passing the sample through the conduit. In some cases, the method further includes quantifying the number of cells that pass through the conduit. For instance, the number of cells of interest may be counted as the cells of interest flow through the conduit. In some instances, cells not of interest are not significantly included in the quantification of the cells of interest.

In certain embodiments, the method includes characterizing the cells as the cells pass through the conduit. A variety of characteristics about the cells may be characterized by the device. For example, characterizing the cells may include determining the average size of the cells, such as the average diameter of the cells. In some instances, characterizing the cells may include determining the shape of the cells.

In some cases, characterizing the cells includes determining the type of cells that are passing through the conduit. For instance, a physical characteristic of the cells of interest may be determined, such as the deformability of the cells. In these embodiments, the method may include determining whether a first population of cells is more or less deformable than other populations of cells (e.g. a second population of cells).

In certain instances, cells to be sorted and characterized are suspended at an appropriate concentration in a suitable liquid medium, e.g., a fluid sample. The fluid sample may include any suitable liquid media (either aqueous or nonaqueous), for example, liquid media such as, but not limited to, water, saline, buffer, organic solvents, cell cultures, animal or human bodily fluids, solutions including particles and/or biological molecules, cellular cytoplasm, cellular extracts, cellular suspensions, solutions of labeled particles or biological molecules, solutions including liposomes, encapsulated material, or micelles, etc. may be used.

A variety of driving mechanisms may be used to produce a flow of the sample fluid through the device. For example, electrophoretic, electrokinetic or electroosmotic forces, or pressure gradients may be used. In some instances, the method includes applying a pressure to the fluid to provide a flow of the fluid through the device. Other embodiments may include pumping the fluid through the device to provide a flow of the fluid through the device. The rate of flow in delivering the fluid sample to the device may be selected to allow sufficient time for the device to sort the cells as they flow through the device. For example, the flow rate may be 0.1 μL/min or more, such as 0.5 μL/min or more, or 1 μL/min or more, or 5 μL/min or more, or 10 μL/min or more, or 25 μL/min or more, or 50 μL/min or more, or 75 μL/min or more, or 100 μL/min or more, or 150 μL/min or more, or 200 μL/min or more. In some instances, the flow rate is 50 μL/min. In some instances, the flow rate is 100 μL/min. In certain embodiments, the method has a detection sensitivity of detecting 1-10 cells of interest (e.g., circulating tumor cells, CTCs) in 7.5 mL of sample.

In some embodiments, the methods include the uniplex analysis of a population of cells in a sample. By “uniplex analysis” is meant that a sample is analyzed to detect the presence of one population of cells in the sample. For example, a sample may include a mixture of cells of interest and other molecular entities that are not of interest. In some cases, the methods include the uniplex analysis of the sample to determine the presence of the cells of interest in the sample mixture.

Certain embodiments include the multiplex analysis of two or more populations of cells in a sample. By “multiplex analysis” is meant that the presence two or more distinct cells, in which the two or more populations of cells are different from each other, is determined. For example, cells may include differences in physical characteristics, such as size and/or deformability. In some instances, the number of populations of cells is greater than 2, such as 3 or more, 4 or more, 5 or more, etc., up to 10 or more, e.g., 20 or more, including 50 or more, distinct populations of cells. In certain embodiments, the methods include the multiplex analysis of 2 to 100 distinct populations of cells, such as 2 to 50 distinct cell populations, including 2 to 10 distinct cell populations.

Methods of the present disclosure also include methods of fabricating the devices described herein. The conduit can be formed by a variety of methods. In some embodiments, the conduit is etched into a substrate which is then sealed by a cover (e.g., an elastomeric cover as described herein). In other embodiments, the conduit can be molded into the cover (e.g., the elastomeric cover as described herein) which is then laid on top of the substrate. In other embodiments, the substrate may be formed into the desired shape using a mold. For example, a substrate precursor solution may be placed into a mold. The substrate precursor solution may include substrate precursor compounds that retain a desired shape when exposed to an external stimulus. For instance, the substrate precursor solution may include substrate precursor monomers that polymerize upon exposure to light, such as UV light. The mold may be removed leaving the conduit, which has retained the desired shape and configuration.

Manufacturing of devices may be carried out by any number of microfabrication techniques. For example, lithographic techniques may be employed in fabricating glass, quartz or silicon substrates, for example, with methods known in the semiconductor manufacturing industries. Photolithographic masking, plasma or wet etching and other semiconductor processing technologies may be used to define microscale elements in and on substrate surfaces. Alternatively, micromachining methods, such as laser drilling, micromilling and the like, may be employed. Similarly, for polymeric substrates, manufacturing techniques such as, but not limited to, injection molding techniques or stamp molding methods may be used. In some cases, large numbers of substrates may be produced using, e.g., rolling stamps to produce large sheets of microscale substrates, or polymer microcasting techniques where the substrate is polymerized within a microfabricated mold.

Exemplary methods of fabricating the present invention are provided herein. It is to be understood that the present invention is not limited to fabrication by one or the other of these methods. Rather, other suitable methods of fabricating the present devices, including modifying the present methods, are also contemplated. One method involves a series of lithographic processes in which the reservoirs and conduit are etched into a planar substrate. These methods can be used to make a large number of devices on a single chip, thus increasing efficiency through parallelization. Another method involves producing a conduit and reservoirs in an elastomeric cover which is then contacted with the substrate.

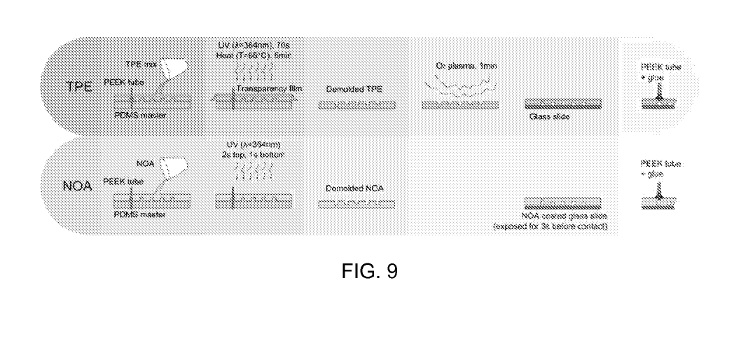

Examples of methods of making a subject device are shown in FIG. 9. For example, a method of making a device using thermoset polyester (TPE) is shown in FIG. 9 (top), where a PDMS master mold is used to form a device using TPE. A tube may be provided to produce a fluid inlet in the TPE device. In some instances, the tube is made of a plastic, such as a thermoplastic polymer, e.g., polyether ether ketone (PEEK). After applying the TPE to the mold, it may be exposed to UV light (e.g., 364 nm light) for a certain period of time (e.g., 70 sec), and/or heat (e.g., 65° C.) for a certain period of time (e.g., 5 min) to polymerize the TPE. The TPE device may be removed from the mold and treated with plasma (e.g., O₂ plasma). A cover, such as a glass slide, may be applied to the TPE to form the conduits. In some instances, a tube may be inserted into the conduit to provide a fluid inlet and/or fluid outlet. A method of making a device using Norland optical adhesive (NOA) is shown in FIG. 9 (bottom), where a PDMS master mold is used to form a device using NOA. A tube may be provided to produce a fluid inlet in the device. In some instances, the tube is made of a plastic, such as a thermoplastic polymer, e.g., polyether ether ketone (PEEK). After applying the NOA to the mold, it may be exposed to UV light (e.g., 364 nm light) for a certain period of time (e.g., 2 sec) on a first side, and exposed to UV light on a second side for a certain period of time (e.g., 1 sec) to polymerize the NOA. The device may be removed from the mold and a cover, such as a glass slide, may be applied to the NOA to form the conduits. In some instances, the cover may be coated with NOA to facilitate bonding of the cover to the NOA. In some instances, a tube may be inserted into the conduit to provide a fluid inlet and/or fluid outlet.

### Systems

Aspects of the present disclosure include a system for sorting cells in a fluid sample. The system includes a device for sorting cells in a fluid sample, as described herein. As described above, the device includes a microfluidic conduit configured to carry a flow of a fluid sample, where the conduit includes a longitudinal divider extending from an interior surface of the conduit towards an opposing interior surface of the conduit, where the longitudinal divider defines a first fluid flow path in fluid communication with a second fluid flow path in the conduit.

The systems of the present disclosure may further include a fluid delivery system, such as a microfluidic or nanofluidic fluid delivery system. Microfluidic fluid delivery systems may include systems where the total volume of biological solution at any one time is 1000 microliters or less. Nanofluidic fluid delivery systems may include systems where the total volume of biological solution at any one time is 1000 nanoliters or less. The fluid deliver system may include one or more pumps configured to provide a flow of a fluid through the device.

Aspects of the presently disclosed system also provide for an integrated “chip” having one or more of the subject microfluidic devices for sorting cells. In certain embodiments, the chip includes a plurality of devices, such as 2 or more devices, or 4 or more devices, or 6 or more, or 8 or more, or 10 or more devices. The two or more devices may be arranged in series (e.g., with a first device positioned upstream from a second device) or in parallel (e.g., with a first and second devices arranged in parallel), or a combination thereof.

In certain embodiments, upstream from the device may be included one or more of a filtration system, a dilution system, and a system to adjust the driving force of the fluid medium. The system may also include an optical detection device for further analytical applications, such as for multiplexed assays or analysis of heterogeneous mixtures. For example, fluorescence of the various cells may be detected as well as the size and type of cells, as described above.

### Utility

The subject devices, systems and methods find use in a variety of different applications where determination of the presence or absence, and/or quantification of one or more cell populations in a sample is desired. In certain embodiments, the methods are directed to the detection of cells in a sample. For example, the methods may be used in the rapid, clinical detection of one or more cells in a biological sample, e.g., as may be employed in the diagnosis of a disease condition in a subject, in the ongoing management or treatment of a disease condition in a subject, etc. In addition, the subject devices, systems and methods may find use in protocols for the detection of cells in a sample for sorting cells of interest from other components of the sample.

In certain embodiments, the subject devices, systems and methods find use in detecting cells. In some cases, the subject devices, systems and methods may be used to detect the presence or absence of particular cells, as well as an increase or decrease in the concentration of particular cells in blood, plasma, serum, or other bodily fluids or excretions, such as but not limited to urine, blood, serum, plasma, saliva, semen, prostatic fluid, nipple aspirate fluid, lachrymal fluid, perspiration, feces, cheek swabs, cerebrospinal fluid, amniotic fluid, gastrointestinal fluid, biopsy tissue, cell lysate samples, and the like. One or more distinct cell populations may be detected. For example, one or more distinct cell populations may be detected, where the cell populations differ from each other by one or more physical characteristic, such as average cell size, deformability

The presence or absence of a type of cell or significant changes in the concentration of a type of cell can be used to diagnose disease risk, presence of disease in an individual, or to tailor treatments for the disease in an individual. For example, the presence of particular cells may influence the choices of drug treatment or administration regimes given to an individual. In evaluating potential drug therapies, the presence and/or amount of a particular cell may be used as a surrogate for a natural endpoint such as survival or irreversible morbidity. If a treatment alters the presence and/or amount of a particular cell, which has a direct connection to improved health, the presence and/or amount of the cells can serve as a surrogate endpoint for evaluating the clinical benefit of a particular treatment or administration regime. Thus, personalized diagnosis and treatment based on the particular cells or panel of cells detected in an individual are facilitated by the subject devices, systems and methods. Furthermore, the early detection of cells associated with diseases is facilitated by the high sensitivity of the subject devices and systems. Due to the capability of detecting multiple cells on a single chip, combined with sensitivity, scalability, and ease of use, the presently disclosed devices, systems and methods finds use in portable and point-of-care or near-patient molecular diagnostics.

In certain embodiments, the subject devices, systems and methods find use in detecting cells for a disease or disease state. In some cases, the disease is a cellular proliferative disease, such as but not limited to, a cancer, a tumor, a papilloma, a sarcoma, or a carcinoma, and the like. For example, the subject devices, systems and methods find use in detecting the presence of a disease, such as a cellular proliferative disease, such as a cancer, tumor, papilloma, sarcoma, carcinoma, or the like. In some instances, the subject devices, systems and methods find use in detecting the presence of one or more populations of cells associated with a disease, such as a cellular proliferative disease, such as a cancer, tumor, papilloma, sarcoma, carcinoma, or the like.

For example, the subject devices, systems and methods may be used to detect and/or quantify acute promylocytic leukemia (APL) cells in a subject. Rapid diagnosis of APL may facilitate earlier administration of treatment protocols to the subject. For example, the subject devices and methods may have an assay time of 15 minutes or less, such as 10 minutes or less, or 7 minutes or less, or 5 minutes or less, or 3 minutes or less, or 2 minutes or less, or 1 minute or less. The subject devices, systems and methods also find use in isolating and screening circulating tumor cells (CTCs) in a subject. In some instances, quantification of CTC levels in a subject may facilitate evaluation and tracking of metastatic progression in the subject. The subject devices, systems and methods may also be used to screen CTCs for specific surface biomarkers, which may facilitate the characterization of the particular CTCs in the subject.

The subject device, systems and methods find use in diagnostic assays, such as, but not limited to, the following: detecting and/or quantifying cells in a sample, as described above; screening assays, where samples are tested at regular intervals for asymptomatic subjects; prognostic assays, where the presence and or quantity of cells of interest are used to predict a likely disease course; stratification assays, where a subject's response to different drug treatments can be predicted; efficacy assays, where the efficacy of a drug treatment is monitored; and the like.

The subject devices, systems and methods also find use in validation assays. For example, validation assays may be used to validate or confirm that a potential disease biomarker is a reliable indicator of the presence or absence of a disease across a variety of individuals. The short assay times for the subject devices, systems and methods may facilitate an increase in the throughput for screening a plurality of samples in a minimum amount of time.

In some instances, the subject devices, systems and methods can be used without requiring a laboratory setting for implementation. In comparison to the equivalent analytic research laboratory equipment, the subject devices and systems provide comparable analytic sensitivity in a portable, hand-held system. In some cases, the weight and operating cost are less than the typical stationary laboratory equipment. The subject systems and devices may be integrated into a single apparatus, such that all the steps of the assay, including detection and sorting of a particle of interest, may be performed by a single apparatus. In addition, the subject systems and devices can be utilized in a home setting for over-the-counter home testing by a person without medical training to detect one or more particles in samples. The subject systems and devices may also be utilized in a clinical setting, e.g., at the bedside, for rapid diagnosis or in a setting where stationary research laboratory equipment is not provided due to cost or other reasons.

### Kits

Aspects of the present disclosure additionally include kits that have a device as described in detail herein. In some instances, the kit includes a packaging for containing the device. In certain embodiments, the packaging may be a sealed packaging, e.g., in a water vapor-resistant container, optionally under an air-tight and/or vacuum seal. In certain instances, the packaging is a sterile packaging, configured to maintain the device enclosed in the packaging in a sterile environment. By “sterile” is meant that there are substantially no microbes (such as fungi, bacteria, viruses, spore forms, etc.). The kits may further include a buffer. For instance, the kit may include a buffer, such as a sample buffer, a wash buffer, and the like. The kits may further include additional reagents, such as but not limited to, detectable labels (e.g., fluorescent labels, colorimetric labels, chemiluminescent labels, multicolor reagents, enzyme-linked reagents, avidin-streptavidin associated detection reagents, radiolabels, gold particles, magnetic labels, etc.), and the like.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., CD, DVD, Blu-Ray, computer-readable memory, etc., on which the information has been recorded or stored. Yet another means that may be present is a website address which may be used via the Internet to access the information at a removed site. Any convenient means may be present in the kits.

As can be appreciated from the disclosure provided above, embodiments of the present invention have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### Example 1

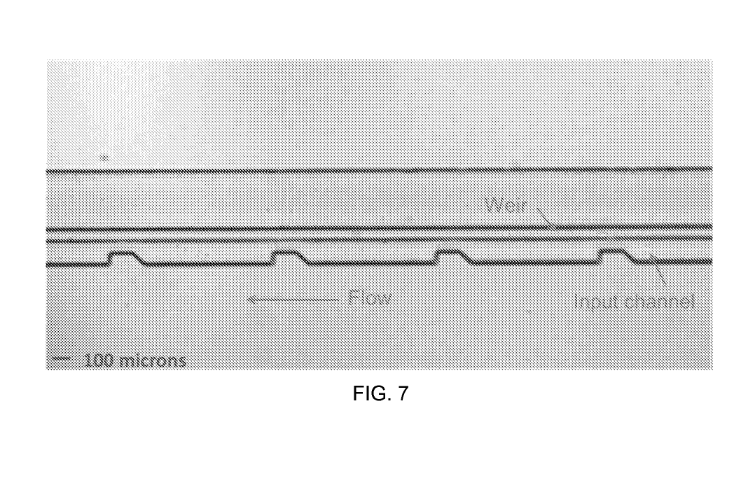

Experiments were performed to develop a low-cost, rapid and efficient, label-free method of isolating and screening circulating tumor cells (CTCs) from the peripheral blood of metastatic cancer patients. The device was a single microfluidic platform that isolated target CTCs based on size without subjecting these cells to high shear forces. See FIG. 7.

Size-Based Isolation of Cancer Cells

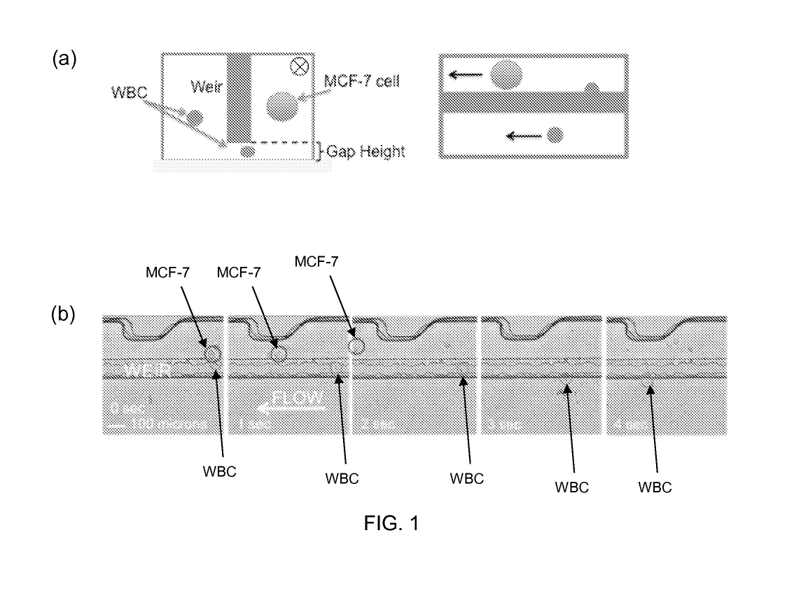

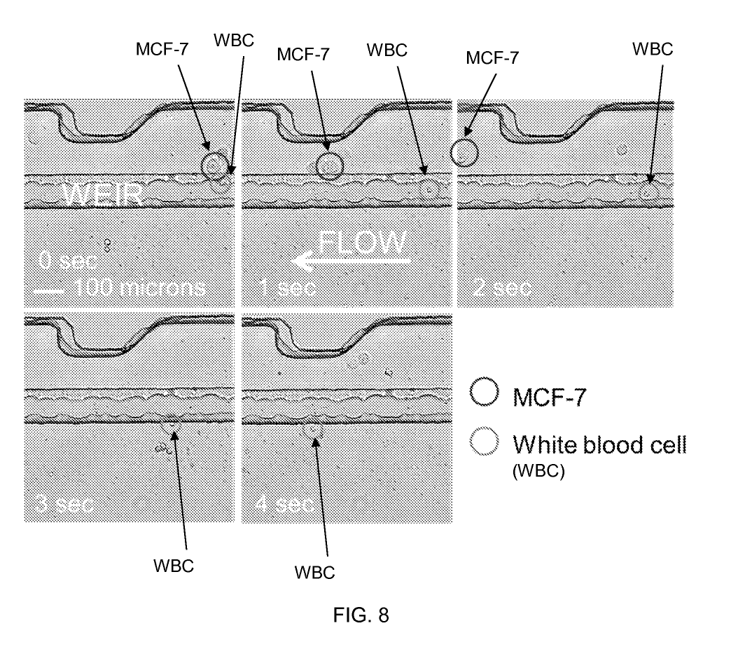

FIG. 1(a) shows a schematic of the device, which included a microfluidic channel that was bilaterally divided by a “weir”, e.g., a divider (also referred to as a wall) suspended above an opposing glass substrate and running parallel to the fluid-flow direction. Soft lithography was used to produce the polydimethylsiloxane (PDMS)-based device. The weir gap height (e.g., the distance between the wall and the opposing glass substrate) was based on the size range and deformability of the cells. As fluid flowed through the device, cells were isolated based on their ability to pass under the weir. FIG. 1(b) and FIG. 8 show images taken at 1 sec intervals of an operating weir device with a 9.0 μm gap height. The device had periodic channel restrictions (e.g., protrusions in the channel) to direct cells toward the weir. The flow rate was 1-50 μL/min. A 2:1 mixture of MCF-7 cells (e.g., breast cancer cell line cells) and white blood cells (WBCs, isolated from healthy human donor blood by lysing RBCs) was suspended in phosphate buffered saline (PBS; 1.50×10⁵ cells/mL) and was flowed through the weir device. As shown in FIG. 1(b), while WBCs moved in a cross-flow direction through the weir gap to the other side of the microchannel, the MCF-7 cells flowed parallel to the weir and did not traverse the weir gap. MCF-7 cells were not observed to move across, or become lodged underneath, the weir. Collection reservoirs downstream on either side of the weir confirmed this result, indicating that 100% recovery of MCF-7 cells was achieved.

As demonstrated above, a subject weir device can be used for isolating metastatic breast-cancer cells spiked in whole blood. The method described herein utilized weirs that were parallel to the microfluidic channel and designed to enrich cancer cell populations using indirect fluid flow and low-shear stresses.

As described above, a 9.0 μm gap height was sufficient for WBCs (average size: 8-15 μm) to deform and pass through the weir gap into the second fluid flow path. MCF-7 cells (average size: 15-25 μm) did not pass through the weir gap and were thus isolated in the initial fluid flow path. The subject devices and methods may also be used (under the same flow conditions and mixed with WBCs) with cells from other breast-cancer cell lines, such as, but not limited to, T47D, SKBR3, and ZR-75-1. The same gap height may be used. Given that different metastatic breast cancer cells may include cells of different size and deformability, a range of gap heights, from 7 to 10 μm, may be used to facilitate high cell recovery. Channel geometry may be modified as well, e.g., elongating the channel to increase the opportunity for as many WBCs to move through the weir gap, thereby increasing the isolated cancer cell concentration. Different gap heights may be provided for different cell types. Thus, devices may include parallel weirs with different gap heights within a single microchannel. As described above, devices composed of PDMS were produced and tested.

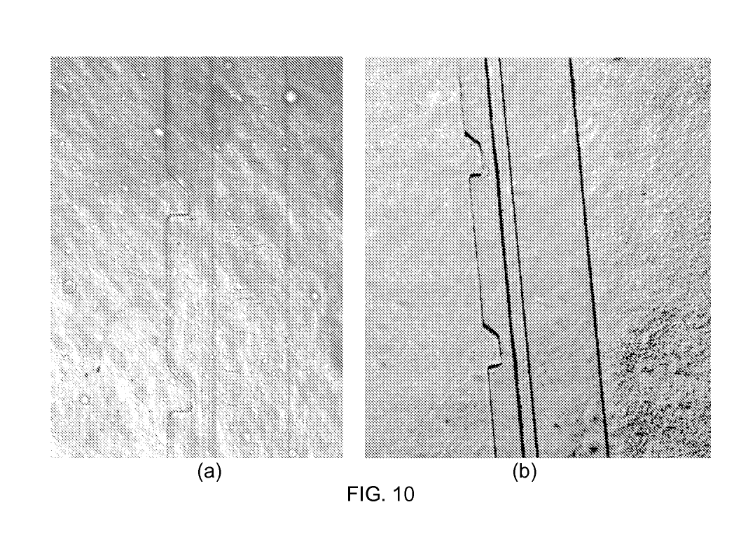

Devices similar to the device shown in FIG. 1(b) were also made from thermoset polyester (TPE, 480λ_{PDMS}, where λ_{PDMS} is the PDMS elastic modulus) and Norland Optical Adhesive 81 (NOA 81, 130λ_{PDMS}), both of which were biocompatible. The devices were fabricated using TPE and NOA 81 with standard lithography techniques. FIG. 10(a) shows an image of a device made of thermoset polyester (TPE), and FIG. 10(b) shows an image of a device made of Norland optical adhesive (NOA 81). Characteristics of the materials used to make the devices are shown in the table below.

Materials for Devices

| | PDMS | TPE | NOA |
|---|---|---|---|
| Bond Pressure Rating | 0.21-0.34 | 1.034 | 0.52 |
| (Mpa) | | | |
| Channel Deformation @ | 15, 20 | <1, <1 | 2.1, 7 |
| 400 μL/min (DW, DH; %) | | | |
| Youngs Modulus (Mpa) | 2.5 | 1200 | 325 |
| Surface Stability | 2 hr | Days | Days |
| (hydrophilicity) | | | |
| Light Transmission | 240-800 | 400-800 | 400-800 |
| (wavelength in nm) | | | |
| Biocompatibility (% cells | 89.6 | 79.2 | 90.6 |
| living at 2 hrs) | | | |
| Solvent Compatibility | − | ++ | + |
| (swelling or feature loss) | | | |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A device for sorting cells in a fluid sample, the device comprising:
a microfluidic conduit configured to carry a flow of a fluid sample and comprising a longitudinal divider extending from an interior surface of the conduit towards an opposing interior surface of the conduit, wherein the longitudinal divider defines a first longitudinal fluid flow path in fluid communication with a second longitudinal fluid flow path in the conduit.

2. The device of claim 1, wherein the conduit comprises a gap between the longitudinal divider and the opposing interior surface of the conduit.

3. The device of claim 2, wherein the gap has a height ranging from 1 μm to 50 μm.

4. The device of claim 1, wherein the first fluid flow path has a width that decreases along the length of the conduit.

5. The device of claim 4, wherein the second fluid flow path has a width that increases along the length of the conduit.

6. The device of claim 1, wherein the conduit comprises an inlet in fluid communication with an upstream end of the first fluid flow path.

7. The device of claim 1, wherein the conduit comprises a first outlet in fluid communication with a downstream end of the first fluid flow path.

8. The device of claim 1, wherein the conduit comprises a second outlet in fluid communication with a downstream end of the second fluid flow path.

9. The device of claim 1, wherein the interior surface of the conduit comprises one or more protrusions extending into the first fluid flow path.

10. A multiplex device for sorting cells in a fluid sample, the device comprising:
a microfluidic conduit configured to carry a flow of a fluid sample and comprising two or more longitudinal dividers each extending from an interior surface of the conduit towards an opposing interior surface of the conduit, wherein the longitudinal dividers define three or more longitudinal fluid flow paths in fluid communication with each other in the conduit.

11. The device of claim 10, wherein the conduit comprises a first gap between a first longitudinal divider and the opposing interior surface of the conduit, and a second gap between a second longitudinal divider and the opposing interior surface of the conduit.

12. The device of claim 11, wherein the first gap has a height greater than the second gap.

13. The device of claim 10, wherein the conduit comprises an inlet in fluid communication with an upstream end of a first fluid flow path.

14. The device of claim 13, wherein the conduit comprises one or more separate fluid outlets each in fluid communication with a downstream end of a different fluid flow path.

15. A method of sorting cells in a fluid sample, the method comprising:
passing a fluid sample comprising a plurality of cells through a microfluidic conduit comprising a longitudinal divider extending from an interior surface of the conduit towards an opposing interior surface of the conduit, wherein the longitudinal divider defines a first longitudinal fluid flow path in fluid communication with a second longitudinal fluid flow path in the conduit.

16. The method of claim 15, further comprising collecting a first population of cells from a first outlet in fluid communication with a downstream end of the first fluid flow path.

17. The method of claim 16, further comprising collecting a second population of cells from a second outlet in fluid communication with a downstream end of the second fluid flow path.

18. The method of claim 15, further comprising quantifying the cells that pass through the conduit.

19. The method of claim 15, further comprising characterizing the cells that pass through the conduit.

20. A kit comprising:
a device comprising:
a microfluidic conduit configured to carry a flow of a fluid sample and comprising a longitudinal divider extending from an interior surface of the conduit towards an opposing interior surface of the conduit, wherein the longitudinal divider defines a first longitudinal fluid flow path in fluid communication with a second longitudinal fluid flow path in the conduit; and
a buffer.
